(19) United States
(12) Patent Application Publication
(10) Pub. No.: US 2026/0174657 A1
(43) Pub. Date: Jun. 25, 2026

(54) COSMETIC CLEANSING CREAM FOR SKIN BRIGHTENING

(71) Applicant: L'OREAL, Paris (FR)

(72) Inventor: Catherine CHIOU, Saddle Brook, NJ (US)

(73) Assignee: L'OREAL, Paris (FR)

(21) Appl. No.: 18/990,788

(22) Filed: Dec. 20, 2024

(51) Int. Cl. A61K 8/49; A61K 8/02; A61K 8/06; A61K 8/365; A61K 8/368; A61K 8/37; A61K 8/46; A61K 8/67; A61K 8/73; A61Q 19/02; A61Q 19/10

(52) U.S. Cl. CPC A61K 8/4926; A61K 8/0212; A61K 8/062; A61K 8/365; A61K 8/368; A61K 8/375; A61K 8/46; A61K 8/466; A61K 8/675; A61K 8/73; A61Q 19/02; A61Q 19/10; A61K 2800/52

(57) ABSTRACT

An oil-in-water emulsion creamy brightening cleanser includes a thiopyridinone compound, a blend of surfactants, at least one thickener, at least one chelator, at least one reducing agent, and a combination of skin care actives. The creamy brightening cleanser is a thick/viscous cream that can be applied as a skin cleanser, a facial mask, or a spot treatment to provide a skin brightening effect that enhances and brightens the appearance of the skin by reducing blemishes, dark spots, uneven skin tone, and/or other skin imperfections.

### TECHNICAL FIELD

The present invention relates to a creamy brightening cleanser in the form of an oil-in-water emulsion. More specifically, it involves a composition that includes a thiopyridinone compound, surfactants, thickeners, chelators, reducing agents, and a combination of skin care actives designed for effective skin cleansing.

### BACKGROUND

Skin cleansing compositions are widely used for personal hygiene and skincare. Traditional formulations often include a variety of surfactants to remove impurities and provide a cleansing effect. However, many existing products can lead to skin irritation or dryness due to harsh chemical components.

Known solutions in the realm of Skin cleansing compositions often focus on the use of surfactants combined with moisturizers or thickeners to mitigate the drying effects. For instance, some products utilize common surfactants like sodium lauryl sulfate, which effectively cleanse but are known to strip the skin of natural oils, leading to dryness and irritation. A significant drawback of these known solutions is their tendency to cause skin irritation or insufficient hydration, particularly when used frequently. Moreover, there is a lack of compositions in the art that can serve multiple purposes, such as being used as a traditional cleanser, a facial mask, or a spot treatment without dripping. Existing products often lack the necessary viscosity and stability to function effectively in multiple applications without compromising user convenience or product efficacy.

Additionally, certain desirable skin care actives, such as antioxidants including thiopyridinone compounds, are challenging to stabilize within formulations. These compounds tend to degrade, losing efficacy over time, especially when exposed to environmental factors like light and air. There is an unmet need for compositions that can effectively stabilize these active ingredients while also providing the aesthetic benefits of cleansing and versatility for multiple application uses, ensuring the actives are present in sufficient quantities to deliver tangible skincare benefits.

The problem addressed by the present invention is the development of a creamy brightening cleanser that achieves effective cleansing while simultaneously maintaining skin moisture and minimizing irritation, stabilizing sensitive active ingredients like thiopyridinone compounds, and being versatile enough to be used as a cleanser, mask, or spot treatment without dripping, which is not adequately resolved by existing formulations.

### SUMMARY

In various embodiments, a creamy brightening cleanser is provided in the form of an oil-in-water emulsion, comprising at least one thiopyridinone compound present in a range from about 0.05% to about 5.0%, and in some embodiments from about 0.3% to about 0.5%, by weight of the creamy brightening cleanser; a blend of surfactants; at least one thickener; at least one chelator; at least one reducing agent; a combination of skin care actives. This composition provides a balanced formulation for effective skin cleansing while maintaining skin hydration. The creamy brightening cleanser has a pH in a range from about 5.5 to about 6.1, wherein the creamy brightening cleanser stabilizes the thiopyridinone compound from photodegradation, evidenced by less than or equal to 50 ppm or no detectable cyclic degradation byproduct from the thiopyridinone compound.

In some embodiments, a creamy brightening cleanser is provided in the form of an oil-in-water emulsion, comprising at least one thiopyridinone compound present in a range from about 0.05% to about 5.0%, or from about 0.1% to about 5%, or from about 0.3% to about 0.5%, by weight of the creamy brightening cleanser; a blend of surfactants; at least one thickener; at least one chelator; at least one reducing agent; a combination of skin care actives, which composition provides a balanced formulation for effective skin cleansing while maintaining skin hydration, the creamy brightening cleanser having a pH in a range from about 5.5 to about 6.1, wherein, after storage at 45° C. for a period of two months, wherein the creamy brightening cleanser stabilizes the thiopyridinone compound from photodegradation, evidenced by less than or equal to 50 ppm or no detectable cyclic degradation byproduct from the thiopyridinone compound and the creamy brightening cleanser demonstrates weight percent loss of the thiopyridinone compound of not more than 10% loss of the thiopyridinone compound, or less than 10%, or less than 5%, or less than 3%, of the thiopyridinone compound based on its original weight in the composition, and maintains viscosity that is not less than about 20,000 cP, or not less than 35,000 cP, or not less than about 50,000 cP, or in a range from about 20,000 cP to greater than 300,000 cP, or in a range from about 35,000 cP to greater than 300,000 cP, or in a range from about 35,000 cP to about 300,000 cP, or in a range from about 30,000 cP to about 50,000 cP.

In some embodiments, the creamy brightening cleanser is a thick/viscous cream that can be applied as a skin cleanser, a facial mask, or a spot treatment. This versatility allows for multiple application methods, enhancing consumer convenience and product utility.

In some embodiments, the creamy brightening cleanser provides a skin brightening effect that enhances and brightens the appearance of the skin by reducing blemishes, dark spots, uneven skin tone, and/or other skin imperfections. This feature offers cosmetic benefits by improving skin appearance.

In some embodiments, the creamy brightening cleanser stabilizes the thiopyridinone compound from photodegradation. This stabilization ensures the longevity and efficacy of the active ingredient within the product.

In some embodiments, the stabilization from photodegradation is evidenced by less than or equal to 50 ppm or no detectable cyclic degradation byproduct from the thiopyridinone compound. This technical advantage guarantees the integrity of the active ingredients, maximizing the product's effectiveness over time.

In some embodiments, the creamy brightening cleanser passes bulk storage stability following an accelerated aging stability protocol. This characteristic ensures the product maintains its quality and performance during storage.

In some embodiments, the thiopyridinone compound comprises 2-mercaptonicotinoyl glycine. This specific compound provides targeted skin benefits and contributes to the overall efficacy of the creamy brightening cleanser.

In some embodiments, the blend of surfactants comprises one or a combination of glyceryl stearate SE, glyceryl stearate citrate, and sodium cocoyl isethionate. This selection of surfactants enhances the cleansing properties of the creamy brightening cleanser while being gentle on the skin.

In some embodiments, the thickener comprises carrageenan or xanthan gum. These thickeners provide the desired viscosity and stability to the creamy brightening cleanser, ensuring ease of application and product consistency.

In some embodiments, the combination of skin care actives comprises one or a combination of antioxidants, vitamins, and beta hydroxy acids. This combination contributes to the skin's health and appearance by providing essential nutrients and promoting cell turnover.

In some embodiments, the beta hydroxy acids are selected from salicylic acid and derivatives thereof, including 5-n-octanoylsalicylic acid, salicylate, sodium salicylate, willow extract, capryloyl salicylic acid, beta hydroxybutanoic acid, propionic acid, beta-hydroxy beta-methylbutyric acid, carnitine tropic acid, and trethocanic acid, and combinations thereof. These acids aid in exfoliating the skin and improving texture and clarity.

In some embodiments, the combination of skin care actives comprises one or a combination of salicylic acid, citric acid, dilauryl thiodipropionate, and niacinamide. This combination provides exfoliating, brightening, and anti-aging benefits, enhancing overall skin health.

In some embodiments, the chelator comprises sodium thiosulfate. This chelator helps to maintain the stability and efficacy of the formulation by binding metal ions that can destabilize active ingredients.

In some embodiments, the reducing agent comprises tetrasodium glutamate diacetate or trisodium ethylenediamine disuccinate. These reducing agents help maintain the integrity of sensitive components within the formulation, contributing to long-term stability.

In some embodiments, the creamy brightening cleanser comprises water and at least one fatty compound. This inclusion provides hydration and nourishment to the skin, enhancing the moisturizing properties of the creamy brightening cleanser.

In some embodiments, the creamy brightening cleanser comprises water-based solvents selected from glycerin and propanediol. These solvents enhance the formulation's texture and help to deliver active ingredients effectively to the skin.

In some embodiments, the fatty compounds comprise one or a combination of cetyl alcohol, hydrogenated jojoba oil, and stearyl alcohol. These fatty compounds contribute to the moisturizing and emollient properties of the creamy brightening cleanser.

In some embodiments, the creamy brightening cleanser comprises one or a combination of additives selected from the group consisting of antimicrobials, preservatives, fillers, penetrants, sequestrants, fragrances, dispersants, and mineral or organic sunscreens. These additives enhance the stability, safety, and sensory attributes of the product.

In some embodiments, a creamy brightening cleanser is provided in the form of an oil-in-water emulsion, comprising at least one thiopyridinone compound comprising 2-mercaptonicotinoyl glycine present in a range from about 0.05% to about 5.0%, or from about 0.3% to about 0.5%, by weight of the creamy brightening cleanser; a blend of surfactants comprising glyceryl stearate SE, glyceryl stearate citrate, and sodium cocoyl isethionate; at least one thickener comprising carrageenan and xanthan gum; at least one chelator comprising sodium thiosulfate; at least one reducing agent comprising tetrasodium glutamate diacetate or trisodium ethylenediamine disuccinate; a combination of skin care actives comprising salicylic acid, citric acid, dilauryl thiodipropionate, and niacinamide; wherein the creamy brightening cleanser comprises water and at least one fatty compound, with water-based solvents selected from glycerin and propanediol, wherein the creamy brightening cleanser is a thick/viscous cream that can be applied as a skin cleanser, a facial mask, or a spot treatment without dripping; wherein the creamy brightening cleanser provides a skin brightening effect that enhances and brightens the appearance of the skin by reducing blemishes, dark spots, uneven skin tone, and/or other skin imperfections; and wherein the creamy brightening cleanser stabilizes the thiopyridinone compound from photodegradation, evidenced by less than or equal to 50 ppm or no detectable cyclic degradation byproduct from the thiopyridinone compound. This comprehensive formulation offers a multifunctional skin care solution that delivers cleansing, moisturizing, and targeted treatment benefits.

The present invention relates to a creamy brightening cleanser in the form of an oil-in-water emulsion. This composition is designed to provide effective cleansing while maintaining skin hydration and stability of active ingredients.

### DETAILED DESCRIPTION

The disclosure relates to compositions for acne care and methods of using the creamy brightening cleansers. According to the invention, the creamy brightening cleanser is provided in a versatile product having a rich creamy texture with a thick creamy consistency for leave on use.

In various embodiments, the creamy brightening cleanser is a gentle skin brightening product provided in a rich creamy format that can be used as one or more of a cleanser for washing, a mask that is left in place for a selected interval, or a leave on spot treatment, each intended for short term use and removal with at least water.

As a mask or facial cleanser to cover large areas, the creamy brightening cleanser demonstrates minimum good spreadability and remains in place without dripping. The creamy brightening cleanser can also be applied and retained without dripping on selected spot areas where brightening is desired. As used herein, “rich creamy texture” refers to a format that is thick/viscous and semi-liquid that clings without running or dripping.

A surprising requirement for stabilization of the thiopyridinone compound, particularly in amounts from about 0.1% to about 5%, for example at about 0.3% of Melasyl, is the inclusion of the blend of surfactants, at least one thickener, at least one chelator, and at least one reducing agent to retain the stabilization of the thiopyridinone compound from degradation, and to maintain phase stability of the creamy brightening cleanser to retain its creamy, thick texture suitable for application as a traditional cleanser, and a non-dripping mask and spot treatment at a pH in a range from about 5.5 to about 6.1. The inventive composition provides a balanced formulation for effective skin cleansing while maintaining skin hydration. As shown in the examples, the inventive composition having a pH in the range from about pH 5.5 to about pH 6.1 demonstrates stability of the thiopyridinone compound characterized by weight percent loss of the thiopyridinone compound of not more than 10% loss of the thiopyridinone compound, or less than 10%, or less than 5%, or less than 3%, of the thiopyridinone compound based on its original weight in the composition after storage at 45° C. for a period of two months. This result is particularly surprising in that the comparative composition having a pH in the range from about pH 5.5 to about pH 6.1 and the same amount of thiopyridinone compound demonstrated a loss of the thiopyridinone compound of more than 76%, and which comparative also demonstrated detectible degraded cyclic compound of thiopyridinone after storage at 45° C. for a period of two months. The physical stability of the inventive composition is characterized by a pale yellow cream with a thick texture that does not break (i.e., no release of droplets, precipitation, cracking or thinning) and maintains viscosity that is not less than about 20,000 cP, or not less than 35,000 cP, or not less than about 50,000 cP, or in a range from about 20,000 cP to greater than 300,000 cP, or in a range from about 35,000 cP to greater than 300,000 cP, or in a range from about 35,000 cP to about 300,000 cP, or in a range from about 30,000 cP to about 50,000 cP after storage at 45° C. for a period of two months. In some embodiments, the inventive composition has a viscosity in a range from about 30,000 cP to about 50,000 cP measured using a Brookfield Ameteck DV-1 Viscometer, T-F #96 Spindle, 10 rpm, 1 min.

### Compositions

### Thiopyridinone Compound:

The creamy brightening cleanser includes at least one thiopyridinone compound, present in a range from about 0.05% to about 5.0%, or from about 0.3% to about 0.5%, by weight. A preferred thiopyridinone compound is 2-mercaptonicotinoyl glycine, commercially known as Melasyl. This compound acts as a skin care active with antioxidant properties, aiding in skin brightening and the reduction of blemishes, dark spots, and uneven skin tone. The stabilization of this compound against photodegradation is crucial for maintaining its efficacy.

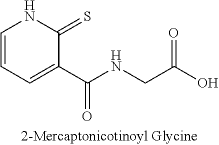 [SMILES: O=C(O)CNC(=O)c1ccc[nH]c1=S]

### 2-Mercaptonicotinoyl Glycine

2-Mercaptonicotinoyl glycine is also known as glycine, N-[(1,2-dihydro-2-thioxo-3-pyridinyl)carbonyl], N-[(1,2-Dihydro-2-thioxo-3-pyridinyl)carbonyl]glycine, and N-[(2-Thioxo-1,2-dihydropyridin-3-yl)carbonyl]glycine, and has the structure shown below.

Generally, 2-Mercaptonicotinoyl glycine provides antioxidant and anti-aging properties to the creamy brightening cleanser. It is formed by combination of nicotinic acid (a form of Vitamin B3) and glycine (an amino acid) with a sulfur-containing thiol group, which contributes to its bioactivity. This unique structure gives it several benefits in the cosmetic application. The thiol (—SH) group in 2-mercaptonicotinoyl glycine acts as a potent antioxidant, scavenging free radicals and protecting the skin from oxidative stress caused by environmental factors such as UV radiation and pollution. The thiol group also has the ability to chelate metal ions, which can help reduce oxidative stress caused by metal-catalyzed reactions in the skin. As a derivative of nicotinic acid, it helps improve skin tone by reducing the appearance of dark spots and pigmentation, contributing to a more even complexion. By combating free radicals and oxidative damage, 2-mercaptonicotinoyl glycine supports collagen preservation and helps reduce the visible signs of aging, such as fine lines and wrinkles.

Referencing WO2023249122, in some embodiments, the about 0.05 to about 5 wt. % of one or more thiopyridinone compounds selected from:
(1) at least one compound selected from compounds of formula (I) below, tautomers of formula (I′) below, salts thereof, solvates, such as hydrates, thereof, optical isomers thereof, racemates thereof, and mixtures thereof:

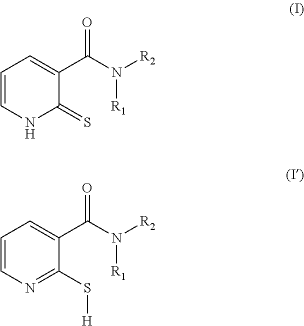

In which Formulas (I) and (I′):
Ri denotes a radical chosen from a) a hydrogen atom, and b) a saturated, linear C1-C10 or branched C3-C10 alkyl group optionally substituted with one or more groups, which may be identical or different, chosen from i) —O—R3, and ii) —S—R3, and
R2 denotes a radical chosen from a) a hydrogen atom, b) a saturated, linear C1-C12 or branched C3-C12 or cyclic C3-C8 hydrocarbonated group optionally substituted with one or more groups, which may be identical or different, chosen from i) —O—R3, ii) —S—R3, iii) —C(O)—O—R₃, and iv) a C5-C12 aryl group optionally substituted with one or more hydroxyls and/or with one or more Ci-Cs alkoxy radicals, and c) a C5-C12 aryl group optionally substituted with one or more hydroxyls and/or with one or more Ci-C«alkoxy radicals, wherein
R3 denotes a radical chosen from a) a hydrogen atom, and b) a saturated, linear C1-C10 or branched C3-C10 alkyl group; and
(2) at least one lipophilic antioxidant; or (2′) a combination of:
at least one antioxidant selected from cinnamic acid or a derivative thereof, hindered phenols or semi-hindered phenols, and thioester antioxidant; e.g., pentaerythrityl tetra-di-t-butyl hydroxyhydrocinnamate, tris(tetramethylhydroxypiperidinol) citrate; and
at least one chelating agent selected from the group consisting of succinic acid and derivatives thereof, phosphor-containing organic acid and derivatives thereof.

It may be preferable that:
Ri of formula (I) and (I′) represents a hydrogen atom; or
Ri of formula (I) and (!′) represents a linear (C1-C10)alkyl group or a branched (C3-C10)alkyl group, especially a linear (Ci-Ce) alkyl group or a branched (C3-C6)alkyl group, such as methyl, ethyl, n-pentyl, n-nonyl, isobutyl, more preferably ethyl, particularly the said alkyl group of Ri is not substituted.

It may be preferable that:
R2 of formula (I) and (I′) represents a hydrogen atom; or
R2 of formula (I) and (I′) represents a linear (C1-C10)alkyl group or a branched (C3-C10)alkyl group, especially a linear (Ci-Ce) alkyl group or a branched (C3-C6)alkyl group, such as methyl, ethyl, n-pentyl, n-nonyl, isobutyl, more preferably methyl or ethyl group; the said alkyl group of R2 being not substituted.

It may be preferable that:
R2 of formula (I) and (I′) represents a linear (C1-C10)alkyl group or a branched (C3-C10)alkyl group, especially a linear (Ci-Ce) alkyl group or a branched (C3-C6)alkyl group, such as methyl, ethyl, n-pentyl, n-nonyl, isobutyl, more preferably methyl or ethyl; the said alkyl group being substituted by one or more groups selected from i), ii), iii) and iv) as defined above, preferably the said alkyl group being substituted by one or two groups selected from i), ii) and iii), more preferably by one or two groups selected from i) and iii), better substituted by one group iii) as carboxy.

It may be preferable that:
R2 of formula (I) and (I′) represents a (C3-C8) cycloalkyl group, preferably a (C5-C7) cycloalkyl group such cyclohexyl; or
R2 of formula (I) and (I′) represents a C5-C12 aryl group optionally substituted with one or more hydroxyls and/or with one or more Ci-Cs alkoxy radicals, preferably a phenyl group particularly not substituted.

It may be preferable that:
R3 of formula (I) and (I′) represents a hydrogen atom; or
R3 of formula (I) and (I′) represents a saturated, linear C1-C10 or branched C3-C10 alkyl group; particularly a linear (Ci-Ce) alkyl group or a branched (C3-C6)alkyl group, preferably a (C1-C4)alkyl group such as methyl group.

It may be more preferable that:
Ri of formula (I) and (I′) represents a radical chosen from a) a hydrogen atom, and b) a saturated, linear Ci-Ce or branched C3-C6 alkyl group optionally substituted with one or more groups, which may be identical or different, chosen from i) —O—R3, and ii) —S—R₃, preferably optionally substituted with one or more groups i);
R2 of formula (I) and (I′) represents a radical chosen from a) a hydrogen atom, and b) a saturated, linear C1-C10 or branched C3-C10 or cyclic C3-C8 such as C5-C6 hydrocarbonated group, optionally substituted with one or more groups, which may be identical or different, chosen from i) —O—R3, ii) —S—R3, iii) —C(O)—O—R3, and iv) a phenyl group optionally substituted with one or more hydroxyls and/or with one or more C1-C4 alkoxy radicals such as methoxy, preferably substituted with one or more groups selected from i) and iii), preferably iii) such as carboxy; and
R3 of formula (I) and (I′) represents a radical chosen from a) a hydrogen atom, and b) a saturated, linear Ci-Ce or branched C3-C6 alkyl group, preferentially, the compounds of formula (I) and tautomer (I′), salts thereof, solvates, such as hydrates, thereof, optical isomers thereof, racemates thereof, and mixtures thereof, have the following meanings:
Ri of formula (I) and (I′) represents a radical chosen from: a) a hydrogen atom, and b) a saturated, linear C1-C4 or branched C3-C4 alkyl group optionally substituted with one or more groups, which may be identical or different, chosen from i) —OR3, more preferably not substituted;
R2 of formula (I) and (I′) represents a radical chosen from a) a hydrogen atom, and b) a saturated, linear C1-C10 or branched C3-C10 or cyclic C3-C8 such as C5-C6 hydrocarbonated group, optionally substituted with one or more groups, which may be identical or different, chosen from i) —O—R3, iii) —C(O)—O—R₃, and iv) a C5-C12 aryl group optionally substituted with one or more hydroxyls and/or with one or more C1-C4 alkoxy radicals; an
R3 of formula (I) and (I′) represents a radical chosen from: a) a hydrogen atom; b) a saturated, linear C1-C4 or branched C3-C4 alkyl group such as methyl or ethyl

The (1) compound may be selected from the compounds 1 to 24 below, tautomers thereof, salts thereof, solvates, such as hydrates, thereof, optical isomers thereof, racemates thereof, and mixtures thereof, particularly the compounds 1, 2, 4, 6, 7, 9, 11, 12, 14, 15, 16, 17, 18, 19, 20, or 21, more particularly 1, 9, 16, 18, 19, 20, or 21, preferably 18, 19, 20, or 21, and more preferably 20:

| No. | Structure | Chemical Name |
|---|---|---|
| 1 | 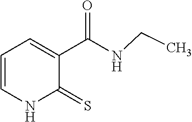 | N-ethyl-2-thioxo-1,2- dihydropyridine-3-carboxamide |
| 2 | 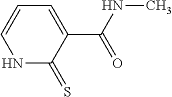 | N-methyl-2-thioxo-1,2- dihydropyridine-3-carboxamide |
| 3 | 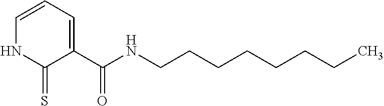 [SMILES: CCCCCCCCNC(=O)c1ccc[nH]c1=S] | N-octyl-2-thioxo-1,2- dihydropyridine-3-carboxamide |
| 4 | 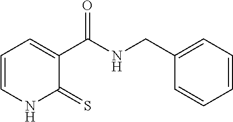 [SMILES: O=C(NCc1ccccc1)c1ccc[nH]c1=S] | N-benzyl-2-thioxo-1,2- dihydropyridine-3-carboxamide |
| 5 | 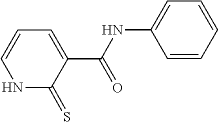 [SMILES: O=C(Nc1ccccc1)c1ccc[nH]c1=S] | N-phenyl-2-thioxo-1,2- dihydropyridine-3-carboxamide |
| 6 | 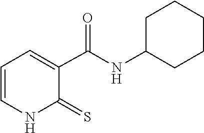 [SMILES: O=C(NC1CCCCC1)c1ccc[nH]c1=S] | N-cyclohexyl-2-thioxo-1,2- dihydropyridine-3-carboxamide |
| 7 | 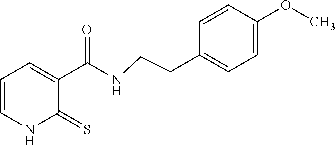 [SMILES: COc1ccc(CCNC(=O)c2ccc[nH]c2=S)cc1] | N-[2-(4-methoxyphenyl)ethyl]- 2-thioxo-1,2-dihydropyridine-3- carboxamide |
| 8 | 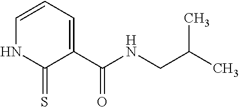 | N-(2-methylpropyl)-2-thioxo- 1,2-dihydropyridine-3- carboxamide |
| 9 | 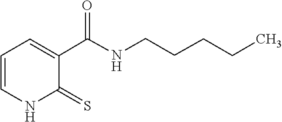 | N-pentyl-2-thioxo-1,2- dihydropyridine-3-carboxamide |
| 10 | 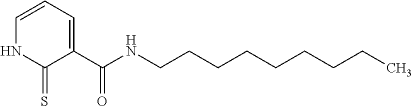 [SMILES: CCCCCCCCCNC(=O)c1ccc[nH]c1=S] | N-nonyl-2-thioxo-1,2- dihydropyridine-3-carboxamide |
| 11 | 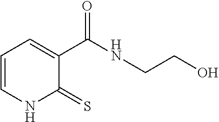 [SMILES: O=C(NCCO)c1ccc[nH]c1=S] | N-(2-hydroxyethyl)-2-thioxo-1,2- dihydropyridine-3-carboxamide |
| 12 | 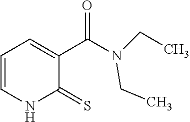 | N,N-diethyl 2-mercaptonicotinamide |
| 13 | 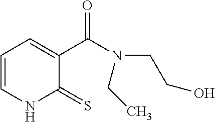 | N-ethyl-N-(2-hydroxyethyl)-2-thioxo- 1,2-dihydropyridine-3-carboxamide |
| 14 | 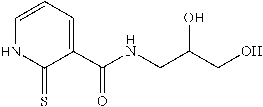 [SMILES: O=C(NCC(O)CO)c1ccc[nH]c1=S] | N-(2,3-dihydroxypropyl)-2-thioxo-1,2- dihydropyridine-3-carboxamide |
| 15 | 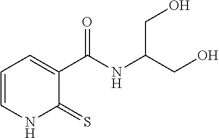 [SMILES: O=C(NC(CO)CO)c1ccc[nH]c1=S] | N-(1,3-dihydroxypropan-2-yl)-2- thioxo-1,2-dihydropyridine-3- carboxamide |
| 16 | 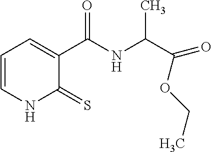 [SMILES: CCOC(=O)C(C)NC(=O)c1ccc[nH]c1=S] | Ethyl N-[(2-thioxo-1,2- dihydropyridin-3-yl)carbonyl]alaninate |
| 17 | 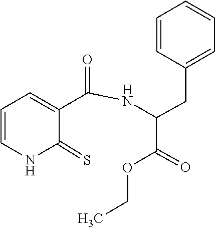 [SMILES: CCOC(=O)C(Cc1ccccc1)NC(=O)c1ccc[nH]c1=S] | Ethyl N-[(2-thioxo-1,2- dihydropyridin-3- yl)carbonyl]phenyl alaninate |
| 18 | 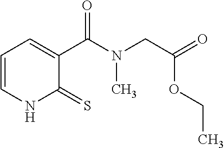 [SMILES: CCOC(=O)CN(C)C(=O)c1ccc[nH]c1=S] | Ethyl N-methyl-N-[(2-thioxo- 1,2-dihydropyridin-3- yl)carbonyl]glycinate |
| 19 | 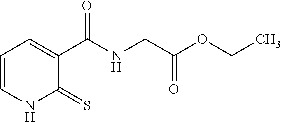 [SMILES: CCOC(=O)CNC(=O)c1ccc[nH]c1=S] | Ethyl N-[(2-thioxo-1,2- dihydropyridin-3- yl)carbonyl]glycinate |
| 20 | 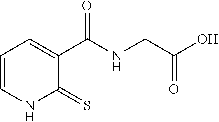 [SMILES: O=C(O)CNC(=O)c1ccc[nH]c1=S] | N-[(2-thioxo-1,2- dihydropyridin-3- yl)carbonyl]glycine |
| 21 | 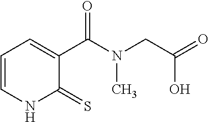 | N-methyl-N-[(2-thioxo-1,2- dihydropyridin-3- yl)carbonyl]glycine |
| 22 | 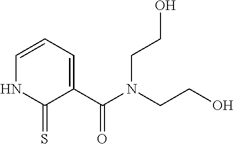 [SMILES: O=C(c1ccc[nH]c1=S)N(CCO)CCO] | N,N-bis(2-hydroxyethyl)-2- thioxo-1,2-dihydropyridine-3- carboxamide |
| 23 | 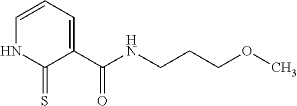 | N-(3-methoxypropyl)-2-thioxo-1,2- dihydropyridine-3-carboxamide |
| 24 | 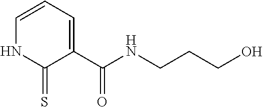 [SMILES: O=C(NCCCO)c1ccc[nH]c1=S] | N-butyl-2-thioxo-1,2-dihydropyridine- 3-carboxamide |

wherein all percentages by weight are based on a total weight of the creamy brightening cleanser.

In some embodiments, the creamy brightening cleanser includes 2-mercaptonicotinoyl glycine, for example, in amounts from about 0.05% to about 5 wt. %, based on the total weight of the cosmetic composition.

In various embodiments, the amount of the at least one thiopyridinone compound is from about 0.05% to about 5%, or from about 0.05% to about 3%, or from about 0.05% to about 2%, or from about 0.05% to about 1%, or from about 0.05% to about 0.8%, or from about 0.1% to about 5%, or from about 0.1% to about 3%, or from about 0.1% to about 2%, or from about 0.1% to about 1%, or from about 0.1% to about 0.8%, or from about 0.3% to about 0.5%, or from about 0.2% to about 5%, or from about 0.2% to about 3%, or from about 0.2% to about 2%, or from about 0.2% to about 1%, or from about 0.2% to about 0.8%, or about 0.3%, or any suitable combination, sub-combination, range, or sub-range thereof by weight, based on the weight of the creamy brightening cleanser. One of ordinary skill in the art, however, will appreciate that other ranges are within the scope of the invention.

Thus, the at least one thiopyridinone compound may be present by weight, based on the total weight of the creamy brightening cleanser, from about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.00, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95, 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 4.90, 4.95 to about 5.00 weight percent, including increments and ranges therein and there between.

### Blend of Surfactants:

The creamy brightening cleanser comprises a blend of surfactants.

In some embodiments, the blend of surfactants includes glyceryl stearate SE, glyceryl stearate citrate, and sodium cocoyl isethionate, each present in a range from about 0.1% to about 20% by weight. Both glyceryl stearate SE and glyceryl stearate citrate function as emulsifying agents to maintain the stability of the oil-in-water emulsion, ensuring the product remains homogeneous and effective throughout its use. Sodium cocoyl isethionate provides a mild cleansing effect ideal for sensitive skin types and contributes to the foaming properties of the product, enhancing the sensory experience during application.

In various embodiments, each surfactant is present in the creamy brightening cleanser from about 0.1% to about 20%, or from about 0.1% to about 15%, or from about 0.1% to about 10%, or from about 0.5% to about 5%, or from about 1% to about 10%, or from about 1% to about 7%, or any suitable combination, sub-combination, range, or sub-range thereof by weight, based on the weight of the creamy brightening cleanser. One of ordinary skill in the art, however, will appreciate that other ranges are within the scope of the invention.

Thus, each surfactant may be present by weight, based on the total weight of the creamy brightening cleanser, from about 0.1, 0.5, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0 to about 20.0 weight percent, including increments and ranges therein and there between.

In some embodiments, when present, sodium cocoyl isethionate is present in the creamy brightening cleanser is from about 1% to about 20%, or from about 2% to about 18%, or from about 5% to about 15%, or from about 8% to about 12%, about 10%, or any suitable combination, sub-combination, range, or sub-range thereof by weight, based on the weight of the creamy brightening cleanser. One of ordinary skill in the art, however, will appreciate that other ranges are within the scope of the invention.

In some embodiments, when present, glyceryl stearate SE is present in the creamy brightening cleanser is from about 1% to about 20%, or from about 2% to about 15%, or from about 3% to about 10%, or from about 4% to about 6%, or about 5%, or any suitable combination, sub-combination, range, or sub-range thereof by weight, based on the weight of the creamy brightening cleanser. One of ordinary skill in the art, however, will appreciate that other ranges are within the scope of the invention.

In some embodiments, when present, glyceryl stearate citrate is present in the creamy brightening cleanser is from about 1% to about 20%, or from about 2% to about 15%, or from about 3% to about 10%, or from about 4% to about 6%, or about 5%, or any suitable combination, sub-combination, range, or sub-range thereof by weight, based on the weight of the creamy brightening cleanser. One of ordinary skill in the art, however, will appreciate that other ranges are within the scope of the invention.

In some embodiments combination of surfactants are present in a total amount in the creamy brightening cleanser from about 1% to about 30%, or from about 10% to about 20%, or any suitable combination, sub-combination, range, or sub-range thereof by weight, based on the weight of the creamy brightening cleanser. One of ordinary skill in the art, however, will appreciate that other ranges are within the scope of the invention.

In various embodiments, the amount of the blend of surfactants is from about 1% to about 5%, or from about 0.5% to about 2%, or from about 1% to about 3%, or any suitable combination, sub-combination, range, or sub-range thereof by weight, based on the weight of the creamy brightening cleanser. One of ordinary skill in the art, however, will appreciate that other ranges are within the scope of the invention. Thus, surfactants may be present by weight, based on the total weight of the creamy brightening cleanser, from about 0.5, 1, 2, 3, 4 to about 5 weight percent, including increments and ranges therein and there between.

In some embodiments, the creamy brightening cleanser may include, or alternatively may be substantially free from, or exclude any one or more of nonionic, cationic, or anionic surfactants. In some embodiments, the creamy brightening cleanser excludes all surfactants except one of sodium cocoyl isethionate, glyceryl stearate SE, or a combination thereof.

In some embodiments, the creamy brightening cleanser may exclude or be devoid of ethylene glycol (PEG) based surfactants or of sulfate/sulfonate based surfactant, or combinations thereof. Some specific but non-limiting examples of surfactants that may be lacking from the creamy brightening cleanser includes those with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di-C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate. Some specific surfactants that are lacking from the cosmetic creamy brightening cleanser include PEG-100 Stearate; PEG-20 Stearate and other esters of Poly(Ethylene Glycol); Sucrose Stearate and other emulsifiers based on sugar esters; Glyceryl Stearate and other glycerol esters; Disodium Ethylene Dicocamide PEG-15 Disulfate; Sodium Steroyl Glutamate and other fatty amides; Steareth-100 and other fatty ethers; Acrylates/C10-30 Alkyl Acrylate Crosspolymer and similar polymeric emulsifiers.

In some embodiments, the creamy brightening cleanser may include or exclude an amphoteric surfactant, for example, betaines, alkyl sultaines, alkyl amphoacetates and alkyl amphodiacetates, alkyl amphoproprionates, amphocarboxylates, alkyl betaines, amidoalkyl betaines, amphophosphates, phosphobetaines, pyrophosphobetaines, carboxyalkyl polyamines, amidoalkyl sultaines, salts thereof, or mixtures thereof.

### Thickener:

The creamy brightening cleanser contains at least one thickener. In some embodiments, the at least one thickener includes carrageenan and xanthan gum, with each thickener present in a range from about 0.1% to about 2% by weight. These thickeners contribute to the viscosity and stability of the creamy brightening cleanser, ensuring it maintains a thick/viscous cream consistency that is suitable for application as a cleanser, mask, or spot treatment. This viscosity helps prevent dripping during use and enhances the user experience by allowing for controlled application.

In various embodiments, the amount of the at least one thickener is from about 0.1% to about 2%, or from about 0.1% to about 1%, or from about 0.5% to about 1.5%, or any suitable combination, sub-combination, range, or sub-range thereof by weight, based on the weight of the creamy brightening cleanser. One of ordinary skill in the art, however, will appreciate that other ranges are within the scope of the invention.

Thus, thickeners may be present by weight, based on the total weight of the creamy brightening cleanser, from about 0.1, 0.2, 0.3, 0.4, 0.5, 0.6, 0.7, 0.8, 0.9, 1, 1.5 to about 2 weight percent, including increments and ranges therein and there between.

### Chelator:

The creamy brightening cleanser contains at least one chelator. In some embodiments, Sodium thiosulfate is used as a chelator in the creamy brightening cleanser, typically present at about 0.01% to 1.0% by weight. It functions by binding metal ions that could destabilize the active ingredients, thus enhancing the overall stability and shelf-life of the product. This chelation process is crucial for preventing oxidation and maintaining the efficacy of sensitive components such as the thiopyridinone compound.

In various embodiments, the amount of sodium thiosulfate is from about 0.01% to about 0.1%, or from about 0.01% to about 0.05%, or from about 0.02% to about 0.08%, or any suitable combination, sub-combination, range, or sub-range thereof by weight, based on the weight of the creamy brightening cleanser. One of ordinary skill in the art, however, will appreciate that other ranges are within the scope of the invention. Thus, sodium thiosulfate may be present by weight, based on the total weight of the creamy brightening cleanser, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9 to about 1.0 weight percent, including increments and ranges therein and there between.

### Reducing Agent:

The creamy brightening cleanser contains at least one reducing agent. In some embodiments, the reducing agent included in the creamy brightening cleanser is either tetrasodium glutamate diacetate or trisodium ethylenediamine disuccinate, present in an amount from about 0.05% to 0.5% by weight. These agents help to maintain the integrity of sensitive components within the formulation by reducing oxidative stress, which contributes to long-term stability and efficacy of the active ingredients.

In various embodiments, the amount of the reducing agent is from about 0.05% to about 0.5%, or from about 0.05% to about 0.3%, or from about 0.1% to about 0.4%, or any suitable combination, sub-combination, range, or sub-range thereof by weight, based on the weight of the creamy brightening cleanser. One of ordinary skill in the art, however, will appreciate that other ranges are within the scope of the invention. Thus, reducing agents may be present by weight, based on the total weight of the creamy brightening cleanser, from about 0.05, 0.1, 0.15, 0.2, 0.25, 0.3, 0.35, 0.4, 0.45 to about 0.5 weight percent, including increments and ranges therein and there between.

### Combination of Skin Care Actives:

The creamy brightening cleanser contains at least one skin care active. In some embodiments, the creamy brightening cleanser features a combination of skin care actives, including salicylic acid, citric acid, dilauryl thiodipropionate, and niacinamide.

Salicylic acid is present in a range from about 0.1% to 2% by weight, providing exfoliation and aiding in acne treatment. Citric acid, included at about 0.1% to 1% by weight, offers skin brightening benefits. Dilauryl thiodipropionate serves as an antioxidant at concentrations from about 0.05% to 0.5% by weight, protecting the skin from environmental damage. Niacinamide, present from about 0.5% to 5% by weight, improves skin texture and barrier function, contributing to the overall health and appearance of the skin.

In various embodiments, the amount of each one of the activities in a combination of skin care actives is from about 1% to about 5%, or from about 0.1% to about 2%, or from about 0.5% to about 4%, or any suitable combination, sub-combination, range, or sub-range thereof by weight, based on the weight of the creamy brightening cleanser. One of ordinary skill in the art, however, will appreciate that other ranges are within the scope of the invention. Thus, skin care actives may be present by weight, based on the total weight of the creamy brightening cleanser, from about 0.1, 0.5, 1, 2, 3, 4 to about 5 weight percent, including increments and ranges therein and there between.

### Water and Solvents:

Water is a primary component, typically making up about 40% to 80% by weight of the creamy brightening cleanser, acting as the medium for the emulsion. The creamy brightening cleanser also includes water-based solvents such as glycerin and propanediol, present in amounts ranging from about 1% to 10% by weight. These solvents enhance skin hydration and aid in the delivery of active ingredients, ensuring they penetrate effectively into the skin for maximum benefit.

In various embodiments, the amount of water is from about 40% to about 80%, or from about 50% to about 65%, or any suitable combination, sub-combination, range, or sub-range thereof by weight, based on the weight of the creamy brightening cleanser. One of ordinary skill in the art, however, will appreciate that other ranges are within the scope of the invention. Thus, water may be present by weight, based on the total weight of the creamy brightening cleanser, from about 40, 45, 50, 55, 60, 65, 70, 75 to about 80 weight percent, including increments and ranges therein and there between.

### Fatty Compounds:

The formulation comprises at least one fatty compound. In some embodiments, the at least one fatty compound could include cetyl alcohol, hydrogenated jojoba oil, and stearyl alcohol. These fatty compounds are included in a range from about 1% to 5% by weight, providing emollient properties that enhance the moisturizing effect of the creamy brightening cleanser. This inclusion ensures that the skin remains soft and supple after use.

In various embodiments, the amount of the fatty compounds is from about 1% to about 5%, or from about 1% to about 3%, or from about 2% to about 4%, or any suitable combination, sub-combination, range, or sub-range thereof by weight, based on the weight of the creamy brightening cleanser. One of ordinary skill in the art, however, will appreciate that other ranges are within the scope of the invention. Thus, fatty compounds may be present by weight, based on the total weight of the creamy brightening cleanser, from about 1, 2, 3, 4 to about 5 weight percent, including increments and ranges therein and there between.

### Optional Additives:

Optionally, the creamy brightening cleanser may contain additives such as antimicrobials, preservatives, fillers, penetrants, sequestrants, fragrances, dispersants, and mineral or organic sunscreens.

In some embodiments, there may be one or more optional actives or other ingredients (herein, “additives”) present in the creamy brightening cleanser, the one or more additives optionally selected from: anti-microbials; chelating agents; oils; fillers; penetrants; sequestrants; fragrances; dispersants; skin care actives such as ceramides, for example, sodium lauroyl lactylate (and) ceramide NP (and) ceramide AP (and) phytosphingosine (and) cholesterol (and) xanthan gum (and) carbomer (and) ceramide EOP; opacifiers; alpha-hydroxy acids; organic and inorganic UV filters; citric acid; phenylethyl resorcinol; hydroxypropyl tetrahydropyrantriol; hydroxyacetophenone; antioxidants, including, but not limited to, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria baicalensis root extract), pine bark extract (Pinus pinaster bark/bud extract), ellagic acid; hyaluronic acid and its derivatives; escin (also known as Aescin, a mixture of saponins with anti-inflammatory, vasoconstrictor and vasoprotective effects found in Aesculus hippocastanum); retinol; niacinamide; and vitamins and vitamin derivatives, such as tocopherol and ascorbic acid; and combinations thereof.

Although the aforementioned optional additives are given as an example, it will be appreciated that other optional components compatible with cosmetic applications known in the art may be used. And of course, any one of the aforementioned additives may be excluded.

In some embodiments, the creamy brightening cleanser includes at least one additive comprising niacinamide present from about 0.1% to about 5%, by weight, based on the weight of the creamy brightening cleanser. In some embodiments, niacinamide is present at about 2%, by weight, based on the weight of the creamy brightening cleanser.

In some embodiments, the creamy brightening cleanser includes at least one additive selected from the group consisting of cetyl alcohol, sodium hydroxide, niacinamide, trisodium ethylenediamine disuccinate, and a combination thereof.

In various embodiments, the amounts of additives, for example, actives and other components, that may be present in the creamy brightening cleanser can range from about 0.001% to about 50%, or from about 0.5% to about 30%, or from about 1.5% to about 20%, and from about 5% to about 15%, or any suitable combination, sub-combination, range, or sub-range thereof by weight, based on the weight of the creamy brightening cleanser.

In some embodiments, the creamy brightening cleanser includes additives comprising cetyl alcohol (2.5%), sodium hydroxide (0.3-0.5%), niacinamide (2%), trisodium ethylenediamine disuccinate (0.5-1%), and pentylene glycol (1%) and butylene glycol (1%).

Thus, one or a combination of additives may be present in the creamy brightening cleanser, by weight, based on the weight of the creamy brightening cleanser, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

### EXAMPLES

The following examples are intended to be non-limiting and explanatory in nature only. In the Examples, amounts are expressed in percentage by weight (wt %) of active materials, relative to the total weight of the creamy brightening cleanser.

### Example 1: Raw Materials

Percentages of each ingredient as may be exemplified in the following composition examples are shown as amount of raw materials, wherein the raw materials may be present in an amount that is equal to the amount of active, or if the raw material has a concentration of active that is less than 100%, then the cosmetic composition includes the raw material that includes active and a suitable solvent, wherein the concentration of active in the raw material is provided herein below in Table 1. When referring to the inventive and comparative examples, it should be presumed that each ingredient as shown in the exemplified composition is final amount of active unless otherwise indicated.

TABLE 1
Select Raw Materials (including RMs having
active concentrations of less than 100%)

| | | % Active |
|---|---|---|
| RM | Description | In RM |
| 2-MERCAPTONICOTINOYL | Melasyl (TM) | 100% |
| GLYCINE | thiopyridinone compound, | |
| | skin depigmentation | |
| SODIUM THIOSULFATE | Sulfur Salt, reducing agent | 100% |
| NIACINAMIDE | Vitamin B3, | 100% |
| | anti-inflammatory | |
| SODIUM COCOYL | Anionic, solid surfactant | 100% |
| ISETHIONATE | | |

### Example 2: Inventive and Comparative Compositions

Table 2 includes inventive compositions and Table 3 shows the stability of Melasyl™ for corresponding inventive examples. Table 4 includes comparative composition and Table 5 includes stability of Melasyl™.

Preparation: Each of the creamy brightening cleansers according to both inventive and comparative examples was prepared by mixing the ingredients shown in Tables 2 and 4 in accordance with the following steps.

The ingredients for Phase A were mixed and heated to 75-80° C. to obtain a mixture of Phase A as a clear liquid. The ingredients for Phase B were mixed and heated to 75-80° C. to obtain a mixture of Phase B as a clear liquid. The mixture of Phase B was added to the mixture of Phase A, and the mixture thus obtained was homogenized with an ESCO Homogenizer at 8,000 rpm at 75-85° C. The ingredients for Phase C were further added to the above mixture obtained in Step 3, followed by homogenizing with the homogenizer at 8,000 rpm at spontaneous temperature for 10 minutes. The ingredients of Phase D were mixed by stirring to obtain a mixture of Phase D. The Mixture of Phase D was added to the above mixture obtained in Step 4, followed by homogenizing with the homogenizer at 8,000 rpm at room temperature for 5 minutes.

TABLE 2
INVENTIVE COMPOSITIONS

| | | Cosmetic | Inventive Examples | | | | |
|---|---|---|---|---|---|---|---|
| Phase | INCI Name | Type | Ex 1 | Ex 2 | Ex 3 | Ex 4 | Ex 5 |
| A | WATER | Solvent | QS to 100% | | | | |
| A | GLYCERIN | Humectant | 20 | 10 | 10 | 10 | 10 |
| A | SALICYLIC ACID | Preservative | 0.45 | 0.45 | 0.45 | 0.45 | 0.45 |
| A | CAPRYLYL GLYCOL | Preservative | | | | | 0.1 |
| A | CARRAGEENAN | Thickener | 1 | | | | |
| A | XANTHAN GUM | Thickener | | 0.5 | 0.5 | 0.5 | 0.5 |
| A | TRISODIUM | Chelator | | 0.3 | 0.3 | 0.3 | 0.3 |
| | ETHYLENEDIAMINE | | | | | | |
| | DISUCCINATE | | | | | | |
| A | TETRASODIUM | Chelator | 0.5 | | | | |
| | GLUTAMATE DIACETATE | | | | | | |
| A | SODIUM THIOSULFATE | Reducer | 0.25 | 0.25 | 0.25 | 0.25 | 0.25 |
| A | SODIUM HYDROXIDE | pH Adjuster | QS to pH 5.5-6.1 | | | | |
| B | GLYCERYL STEARATE SE | O/W | 7.5 | 7.5 | 10 | 7.5 | 10 |
| | | emulsifier | | | | | |
| B | GLYCERYL STEARATE | O/W | | 2.5 | 2.5 | 2.5 | 2.5 |
| | CITRATE | emulsifier | | | | | |
| B | CETYL ALCOHOL | Fatty | 3.8 | 3.8 | | 3.8 | |
| | | Compound | | | | | |
| B | HYDROGENATED JOJOBA | Fatty | | | | 1 | |
| | OIL | Compound | | | | | |
| B | STEARYL ALCOHOL | Fatty | | | 4 | | 4 |
| | | Compound | | | | | |
| B | DILAURYL | Antioxidant | | | | | 0.2 |
| | THIODIPROPIONATE | | | | | | |
| C | SODIUM COCOYL | Solid | 5 | 10 | 10 | 10 | 10 |
| | ISETHIONATE | Surfactant | | | | | |
| D | WATER | Solvent | 10 | 10 | 10 | 10 | 10 |
| D | NIACINAMIDE | Vitamin B3 | 5 | 5 | 5 | 5 | 5 |
| D | 2-MERCAPTONICOTINOYL | Melasyl | 0.3 | 0.3 | 0.3 | 0.3 | 0.3 |
| | GLYCINE | (TM) | | | | | |

| Total Wt % | 100 | 100 | 100 | 100 | 100 |
|---|---|---|---|---|---|
| Appearance: | Pale yellow cream | | | | |
| pH (at 25° C.): | 5.5~6.1 | | | | |
| Viscosity (Brookfield)*, cP: | 35,000 | 40,000 | 48,000 | 45,000 | 32,500 |
| Bulk Stability at 45° C. for 2 months: | PASS | PASS | PASS | PASS | PASS |

*Brookfield Ameteck DV-1 Viscometer, T-F #96 Spindle, 10 rpm, 1 min

TABLE 3
Stability of Melasyl (wt %)

| Inventive Examples | Ex 1 | Ex 2 | Ex 3 | Ex 4 | Ex 5 |
|---|---|---|---|---|---|
| T0 RT: | 0.310% | 0.326% | 0.303% | 0.328% | 0.290% |
| T8 w 45° C.: Remaining | 0.296% | 0.316% | 0.308% | 0.318% | 0.302% |
| thiopyridinone compound | | | | | |
| % Loss of thiopyridinone | 4.52% | 3.07% | 1.65% | 3.05% | 4.14% |
| compound | | | | | |
| T8 w 45° C.: cyclic | <50 | <50 | <50 | <50 | <50 |
| degradation product* | ppm | ppm | ppm | ppm | ppm |

*Detection limit at 50.0 ppm

TABLE 4
COMPARATIVE COMPOSITION, Example 6

| | | Ex. 6 |
|---|---|---|
| Phase | INCI Name | wt % |
| A1 | WATER/AQUA (QS) | QS to 100% |
| A1 | MAGNESIUM ALUMINUM SILICATE | 3 |
| A1 | XANTHAN GUM | 0.5 |
| A2 | GLYCERIN | 7 |
| A2 | BUTYLENE GLYCOL | 1 |
| A2 | PENTYLENE GLYCOL | 1 |
| A2 | SALICYLIC ACID | 0.45 |
| A2 | LACTIC ACID (90% active) | 2.22 |
| A2 | GLUCONOLACTONE | 2 |
| A2 | TRISODIUM ETHYLENEDIAMINE | 1 |
| | DISUCCINATE | |
| A2 | SODIUM THIOSULFATE | 0.25 |
| A3 | SODIUM HYDROXIDE (QS to pH 5.5-6.1) | QS |
| B | GLYCERYL STEARATE SE | 5 |
| B | CETYL ALCOHOL | 2.5 |
| C | SODIUM COCOYL ISETHIONATE | 10 |
| C | KAOLIN | 10 |
| D | WATER | 10 |
| D | NIACINAMIDE | 2 |
| D | 2-MERCAPTONICOTINOYL GLYCINE | 0.3 |
| | Total (wt %): | 100 |
| | Appearance: | Pale yellow |
| | | paste |
| | pH (at 25° C.): | 5.81 |
| | Viscosity (Brookfield)*, cP: | 170,000 |
| | Bulk Stability at 45° C. for 2 months: | PASS |

*Brookfield Ameteck DV-1 Viscometer, T-F #96 Spindle, 10 rpm, 1 min

TABLE 5
Stability of Melasyl (wt %) in Comparative Example 6

| Inventive Examples | Ex 6 |
|---|---|
| T0 RT: | 0.301% |
| T8 w 45° C.: Remaining thiopyridinone compound | 0.071% |
| % Loss of thiopyridinone compound | 76.4% |
| T8 w 45° C.: cyclic degradation product* | 70.0 ppm |

*Detection limit at 50.0 ppm

As can be seen from Tables 4 and 5, all examples have passed the bulk stability test at 45° C. for 2 months. Further, the depigmentation active ingredient, Melasyl, was monitored for its weight percentage in the formula under the same stability test protocol. All measurements of loss of the thiopyridinone compound are expressed as wt % loss compared to its original weight % in the total composition. For instance, when dosed at 0.3% (by wt) of total composition, a 5% or less loss of the compound means that the final measured Melasyl should not be less than 0.285% of total composition [0.3%×95%=0.285%] after stored at 45° C. for 2 months. According to the invention, a stable composition will demonstrate not more than 10% loss of the thiopyridinone compound, or less than 10%, or less than 5%, or less than 3%, of the thiopyridinone compound based on its original weight in the composition.

All Inventive Examples (Ex 1.˜Ex 5) contain remaining Melasyl at the end of 45° C. for 2 months with weight percent loss of less than 5% by weight based on the original weight percent of the compound in the creamy brightening cleanser. In sharp contrast, the Melasyl compound in Comparative Example 6 is not stable and has dropped from the initial 0.3% to 0.071%, i.e. a total loss of more than 76% of its original weight in the composition. Further, a degraded cyclic compound of thiopyridinone was detected. Thus Comparative Example 6 was not suited for the current invention.

The physical stability of the inventive composition is characterized by a pale yellow cream with a thick texture that does not break (i.e., no release of droplets, precipitation, cracking or thinning) and maintains viscosity that is not less than about 20,000 cP, or not less than 35,000 cP, or not less than about 50,000 cP, or in a range from about 20,000 cP to greater than 300,000 cP, or in a range from about 35,000 cP to greater than 300,000 cP, or in a range from about 35,000 cP to about 300,000 cP, or in a range from about 30,000 cP to about 50,000 cP, after storage at 45° C. for a period of two months. Generally, the inventive composition has a viscosity in a range from about 30,000 cP to about 50,000 cP measured using a Brookfield Ameteck DV-1 Viscometer, T-F #96 Spindle, 10 rpm, 1 min.

While the disclosure has been described with reference to specific embodiments, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

### Definitions and Terms

In the context of this description, the following definitions apply. These definitions are intended to clarify the terms used in the description and claims, providing a comprehensive understanding of the invention's scope and applications.

“Oil-in-water emulsion” refers to a type of emulsion where oil droplets are dispersed in a continuous water phase, providing a stable formulation that is easily applied to the skin.

“Thiopyridinone compound” encompasses compounds containing a thiopyridinone moiety, known for their antioxidant properties and ability to enhance skin appearance by reducing imperfections.

“Surfactants” are surface-active agents that lower the surface tension between two substances, such as oil and water, aiding in the formation of stable emulsions and effective cleansing.

“Thickener” refers to ingredients that increase the viscosity of the creamy brightening cleanser, providing a desirable texture and stability without affecting the active components' efficacy.

“Chelator” is a compound that binds to metal ions, preventing them from catalyzing the degradation of other ingredients within the formulation, thus enhancing stability.

“Reducing agent” refers to compounds that prevent oxidation of sensitive ingredients, thereby maintaining the product's integrity and effectiveness over time.

“Skin care actives” are ingredients included in the formulation to provide specific benefits to the skin, such as exfoliation, brightening, and improvement of texture and tone.

“Fatty compounds” are lipophilic substances that provide moisturizing properties and enhance the emollience of the creamy brightening cleanser, contributing to skin softness and hydration.

“Additives” include a range of optional components such as preservatives, fragrances, and UV filters, which enhance the overall sensory experience and stability of the product.

The term “physiologically acceptable medium” is intended to mean a medium that is compatible with human keratin materials such as the skin of the body or of the face, the lips, the mucous membranes, the eyelashes, the nails, the scalp and/or the hair.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). As used herein, the terms “comprising,” “having,” and “including” (or “comprise,” “have,” and “include”) are used in their open, non-limiting sense. The term “comprising” is intended to be inclusive or open-ended and does not exclude any additional, unrecited element, method, step, or material.

The term “consisting of” excludes any element, step, or material other than those specified in the claim and, in the latter instance, impurities ordinary associated with the specified material(s).

The term “consisting essentially of” limits the scope of a claim to the specified elements, steps, or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point.

A range given of “about 3% to 7%” is intended to have the term “about” modifying both the 3% and the 7% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient in the raw material except as specifically stated. All amounts given herein are relative to the amount of active material, unless otherwise indicated.

All percentages, parts and ratios herein are based upon the total weight of embodiments of the creamy brightening cleanser of the present disclosure, unless otherwise indicated.

As used herein, the term “surfactants,” as well as any specifically identified surfactants, includes salts of the surfactants even if not explicitly stated.

As used herein, the term “synthetic” means a material that is not of natural origin. The term “natural” and “naturally-sourced” and “nature-based” means a material of natural origin, such as derived from plants, which also cannot be subsequently chemically or physically modified. “Plant-based” means that the material came from a plant.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

As used herein, the term “substantially free” or “essentially free” as used herein means the specific material may be present in small amounts that do not materially affect the basic and novel characteristics of the embodiments of the creamy brightening cleanser according to the disclosure or the material may be absent. For instance, there may be less than 2% by weight of a specific material added to a composition, based on the total weight of the creamy brightening cleansers (provided that an amount of less than 2% by weight does not materially affect the basic and novel characteristics of embodiments of the creamy brightening cleanser according to the disclosure. Similarly, the creamy brightening cleansers may include less than 2%, less than 1.5%, less than 1%, less than 0.5%, less than 0.1%, less than 0.05%, or less than 0.01%, or none (0%) of the specified material. Furthermore, all components that are positively set forth in the instant disclosure may be negatively excluded from the claims, e.g., a claimed composition may be “free,” “devoid,” “essentially free” (or “substantially free”) of one or more components that are positively set forth in the instant disclosure and a component or ingredient may be excluded from the composition. The term “substantially free” or “essentially free” as used herein may also mean that the specific material is not added to the creamy brightening cleanser but may still be present in a raw material that is included in the creamy brightening cleanser.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A creamy brightening cleanser in the form of an oil-in-water emulsion, comprising:
(a) at least one thiopyridinone compound present in a range from about 0.05% to about 5.0% by weight of the creamy brightening cleanser;
(b) a blend of surfactants;
(c) at least one thickener;
(d) at least one chelator;
(e) at least one reducing agent;
(f) a combination of skin care actives
wherein the creamy brightening cleanser stabilizes the thiopyridinone compound from photodegradation, evidenced by less than or equal to 50 ppm or no detectable cyclic degradation byproduct from the thiopyridinone compound.

2. The creamy brightening cleanser according to claim 1, wherein, after storage at 45° C. for a period of two months, the creamy brightening cleanser demonstrates weight percent loss of the thiopyridinone compound of less than 10% based on its original weight in the composition, and a stable viscosity in a range from about 30,000 cP to about 50,000 cP

3. The creamy brightening cleanser according to claim 1, wherein the creamy brightening cleanser is a thick/viscous cream that can be applied as a skin cleanser, a facial mask, and/or a spot treatment.

4. The creamy brightening cleanser according to claim 1, wherein the creamy brightening cleanser provides a skin brightening effect that enhances and brightens the appearance of the skin by reducing blemishes, dark spots, uneven skin tone, and/or other skin imperfections.

5. The creamy brightening cleanser according to claim 1, wherein the creamy brightening cleanser stabilizes the thiopyridinone compound from photodegradation.

6. The creamy brightening cleanser according to claim 5, wherein the stabilization from photodegradation is evidenced by less than or equal to 50 ppm or no detectable cyclic degradation byproduct from the thiopyridinone compound.

7. The creamy brightening cleanser according to claim 1, wherein the creamy brightening cleanser passes bulk storage stability following an accelerated aging stability protocol.

8. The creamy brightening cleanser according to claim 1, wherein the thiopyridinone compound comprises 2-mercaptonicotinoyl glycine.

9. The creamy brightening cleanser according to claim 1, wherein the blend of surfactants comprises one or a combination of glyceryl stearate SE, glyceryl stearate citrate, and sodium cocoyl isethionate.

10. The creamy brightening cleanser according to claim 1, wherein the thickener comprises carrageenan or xanthan gum.

11. The creamy brightening cleanser according to claim 1, wherein the combination of skin care actives comprises one or a combination of antioxidants, vitamins, and beta hydroxy acids.

12. The creamy brightening cleanser according to claim 11, wherein the beta hydroxy acids are selected from salicylic acid and derivatives thereof, including 5-n-octanoylsalicylic acid, salicylate, sodium salicylate, willow extract, capryloyl salicylic acid, beta hydroxybutanoic acid, propionic acid, beta-hydroxy beta-methylbutyric acid, carnitine tropic acid, and trethocanic acid, and combinations thereof.

13. The creamy brightening cleanser according to claim 1, wherein the combination of skin care actives comprises one or a combination of salicylic acid, citric acid, dilauryl thiodipropionate, and niacinamide.

14. The creamy brightening cleanser according to claim 1, wherein the chelator comprises sodium thiosulfate.

15. The creamy brightening cleanser according to claim 1, wherein the reducing agent comprises tetrasodium glutamate diacetate or trisodium ethylenediamine disuccinate.

16. The creamy brightening cleanser according to claim 1, wherein the creamy brightening cleanser comprises water and at least one fatty compound.

17. The creamy brightening cleanser according to claim 16, wherein the creamy brightening cleanser comprises water-based solvents selected from glycerin and propanediol.

18. The creamy brightening cleanser according to claim 16, wherein the fatty compounds comprise one or a combination of cetyl alcohol, hydrogenated jojoba oil, and stearyl alcohol.

19. The creamy brightening cleanser according to claim 1, wherein the creamy brightening cleanser comprises one or a combination of additives selected from the group consisting of antimicrobials, preservatives, fillers, penetrants, sequestrants, fragrances, dispersants, and mineral or organic sunscreens.

20. A creamy brightening cleanser in the form of an oil-in-water emulsion, comprising:
(a) at least one thiopyridinone compound comprising 2-mercaptonicotinoyl glycine present in a range from about 0.3% to about 0.5%, by weight of the creamy brightening cleanser;
(b) a blend of surfactants comprising glyceryl stearate SE, glyceryl stearate citrate, and sodium cocoyl isethionate;
(c) at least one thickener comprising carrageenan and xanthan gum;
(d) at least one chelator comprising sodium thiosulfate;
(e) at least one reducing agent comprising tetrasodium glutamate diacetate or trisodium ethylenediamine disuccinate;
(f) a combination of skin care actives comprising salicylic acid, citric acid, dilauryl thiodipropionate, and niacinamide;
wherein the creamy brightening cleanser comprises water, at least one fatty compound, and at least one water-based solvent selected from glycerin and propanediol,
wherein the creamy brightening cleanser is a thick/viscous cream that can be applied as a skin cleanser, a facial mask, or a spot treatment;
wherein the creamy brightening cleanser provides a skin brightening effect that enhances and brightens the appearance of the skin by reducing blemishes, dark spots, uneven skin tone, and/or other skin imperfections; and
wherein the creamy brightening cleanser stabilizes the thiopyridinone compound from photodegradation, evidenced by less than or equal to 50 ppm or no detectable cyclic degradation byproduct from the thiopyridinone compound.
